(19) United States
(12) Patent Application Publication
(10) Pub. No.: US 2015/0204890 A1
(43) Pub. Date: Jul. 23, 2015

(54) MICROARRAYS OF G PROTEIN COUPLED RECEPTORS

(71) Applicant: PRINCETON INSTITUTE OF LIFE SCIENCES, Princeton, NJ (US)

(72) Inventors: Alexey Ryazanov, Princeton, NJ (US); Alexander V. Chikunov, Moscow (RU)

(21) Appl. No.: 14/413,572

(22) PCT Filed: Jul. 8, 2013
(86) PCT No.: PCT/US13/49531

§ 371 Date: Jan. 8, 2015

Related U.S. Application Data
(60) Provisional application No. 61/669,184, filed on Jul. 9, 2012.

(51) Int. Cl. G01N 33/74

(52) U.S. Cl. CPC G01N 33/74; G01N 2500/04; G01N 2333/726

(57) ABSTRACT

Microarrays encompassing GPCRs, methods of making such microarrays, and methods of using such microarrays are described.

### CROSS REFERENCE TO RELATED APPLICATIONS

The present application claims the benefit of U.S. provisional application Ser. No. 61/669,184, filed on Jul. 9, 2012, the contents of which are herein incorporated by reference in their entirety.

### BACKGROUND

In humans, about 400 known different G protein coupled receptors (GPCRs) exist for endogenous ligands that regulate physiological processes by mediating effects of hormones, neurotransmitters, peptides, metabolites, nucleotides and ions. GPCRs are a major target in the development of pharmaceutical products, including selective GPCR agonists and antagonists that activate or inhibit specific GPCRs.

### SUMMARY

The present disclosure provides the discovery that microarrays of GPCRs, e.g., microarrays of the human receptorome, can be used to analyze simultaneously and directly the activation of GPCRs by agents, e.g., therapeutic agents. The present invention therefore provides microarrays of GPCRs and methods of making and using such microarrays, such as to identify particular GPCRs activated by agents. In some embodiments, the present invention provides methods of identifying and/or characterizing GPCR modulating agents, and in particular provides methods of determining and/or detecting a GPCR modulation fingerprint for one or more particular agents; in some such embodiments such a GPCR modulation fingerprint includes information on effects of the agent(s) on all GPCRs in a provided microarray, e.g., all GPCRs in a human receptorome.

In some aspects, the invention features a microarray comprising or consisting of a substrate and at least about 400 unique G protein coupled receptors (GPCR) disposed on the substrate, wherein each GPCR is disposed at a predetermined location of the substrate, wherein each GPCR is linked to a first marker and a second marker, and wherein the first marker and the second marker produce a GPCR conformation dependent detectable signal.

In some aspects, the invention features a method of producing a microarray, comprising: providing at least about 400 different GPCRs, wherein each GPCR is linked to a first marker and a second marker, and wherein the first marker and the second marker produce a GPCR conformation dependent detectable signal; and disposing each GPCR at a predetermined location of the substrate.

In some aspects, the invention features a method of identifying one or more GPCRs activated by a ligand, comprising: providing a microarray comprising or consisting of a substrate and at least about 400 different GPCRs disposed on the substrate, wherein each GPCR is disposed at a predetermined location of the substrate, wherein each GPCR is linked to a first marker and a second marker, and wherein the first marker and the second marker produce a GPCR conformation dependent detectable signal; contacting the GPCRs with the ligand; and measuring a presence or absence of a GPCR conformation dependent detectable signal at a predetermined location of the substrate, wherein a detectable signal measured at a predetermined location of the substrate identifies the GPCR at the predetermined location as a GPCR activated by the ligand.

In some aspects, the invention features a method of identifying an agonist of one or more GPCRs, comprising: providing a microarray comprising or consisting of a substrate and at least about 400 different GPCRs disposed on the substrate, wherein each GPCR is disposed at a predetermined location of the substrate, wherein each GPCR is linked to a first marker and a second marker, and wherein the first marker and the second marker produce a GPCR conformation dependent detectable signal; measuring an initial absence of a GPCR conformation dependent detectable signal at one or more predetermined locations of the substrate; contacting the GPCRs with a test agent; and measuring one or more GPCR conformation dependent detectable signals from the microarray in the presence of the test agent, wherein measurement of one or more GPCR conformation dependent detectable signals from the one or more predetermined locations of the substrate identifies the test agent as an agonist of a GPCR at the one or more predetermined locations.

In some aspects, the invention features a method of identifying an antagonist of one or more GPCRs, comprising: providing a microarray comprising or consisting of a substrate and at least about 400 different GPCRs disposed on the substrate, wherein each GPCR is disposed at a predetermined location of the substrate, wherein each GPCR is linked to a first marker and a second marker, and wherein the first marker and the second marker produce a GPCR conformation dependent detectable signal; contacting the GPCRs with an agonist; measuring an initial GPCR conformation dependent detectable signal at one or more predetermined locations of the substrate in the presence of the agonist; contacting the GPCRs with a test agent; and measuring an absence of one or more GPCR conformation dependent detectable signals from the microarray in the presence of the test agent, wherein measurement of an absence of one or more GPCR conformation dependent detectable signals from the one or more predetermined locations of the substrate identifies the test agent as an antagonist of a GPCR at the one or more predetermined locations.

In some aspects, the invention features a method of identifying an inverse agonist of one or more GPCRs, comprising: providing a microarray comprising or consisting of a substrate and at least about 400 different GPCRs disposed on the substrate, wherein each GPCR is disposed at a predetermined location of the substrate, wherein each GPCR is linked to a first marker and a second marker, and wherein the first marker and the second marker produce a GPCR conformation dependent detectable signal; measuring an initial GPCR conformation dependent detectable signal at one or more predetermined locations of the substrate; contacting the GPCRs with a test agent; and measuring an absence of one or more GPCR conformation dependent detectable signals from the microarray in the presence of the test agent, wherein measurement of an absence of one or more GPCR conformation dependent detectable signals from the one or more predetermined locations of the substrate identifies the test agent as an inverse agonist of a GPCR at the one or more predetermined locations.

### BRIEF DESCRIPTION OF THE DRAWINGS

The following figures are presented for the purpose of illustration only, and are not intended to be limiting.

FIG. 1 is a schematic illustration of an exemplary nanodisc incorporating a GPCR.

FIG. 2 is a schematic diagram of an exemplary method of detecting GPCR activation by fluorescence resonance energy transmission (FRET).

FIG. 3A is a schematic illustration of an exemplary cell-free method of expressing GPCRs based on constant exchange. FIG. 3B is a schematic illustration of an exemplary reactor for expressing GPCRs based on constant exchange cell-free methods.

FIG. 4 is a schematic overview of an exemplary method of expressing GPCRs in a cell-free system and preparing a microarray.

FIG. 5 is a flow diagram of an exemplary FRET microarray analyzer.

FIG. 6 is a schematic representation of an exemplary microarray drug analysis.

All publications, patent applications, patents, and other references mentioned herein, including GenBank database sequences, are incorporated by reference in their entirety. In case of conflict, the present specification, including definitions, will control. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

Other features and advantages of the invention will be apparent from the following detailed description, and from the claims.

### DEFINITIONS

In order for the present invention to be more readily understood, certain terms are first defined below. Additional definitions for the following terms and other terms are set forth throughout the specification.

Agonist: As used herein, an “agonist” is a ligand that binds to a GPCR so that an increase in a signaling activity is observed relative to that of the GPCR in an unliganded (unbound) state. In some embodiments, an agonist interacts with all or part of a GPCR structure, e.g., a GPCR structure involved in binding one or more naturally-occurring (e.g., reference) ligands that bind to the GPCR. In some embodiments, an agonist interacts with all or part of a GPCR structure, e.g., a GPCR structure involved in binding one or more naturally-occurring (e.g., reference) ligands that regulate GPCR activity in vivo. In some embodiments, an agonist competes with one or more naturally-occurring (e.g., reference) ligands (i.e., a naturally-occurring ligand that binds to and/or regulates activity of) of the GPCR for binding to the GPCR. In some embodiments, binding by an agonist stabilizes one or more GPCR conformations (e.g., conformations that promote and/or participate in signaling).

Amino acid entity: As used herein, an “amino acid entity” is any compound or substance that can be incorporated into a polypeptide chain without terminating or significantly disrupting a polypeptide chain. In some embodiments, an amino acid entity is incorporated into a polypeptide chain through participation in one or more peptide bonds. In some embodiments, an amino acid entity is a naturally-occurring amino acid (e.g., arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine). In some embodiments, an amino acid entity is a synthetic amino acid entity. In some embodiments, an amino acid entity is a D-amino acid. In some embodiments, an amino acid entity is an L-amino acid. In some embodiments, an amino acid entity is a modified, e.g., chemically modified, amino acid. In some embodiments, an amino acid entity comprises one or more additional methyl, amino, or acetyl groups as compared with a reference amino acid (e.g., a naturally-occurring amino acid).

Antagonist: As used herein, an “antagonist” is a ligand that binds to a GPCR so that a decrease in a signaling activity of the GPCR is observed relative to that observed upon binding of an agonist of the GPCR to the GPCR. In some embodiments, an antagonist interacts with all or part of a GPCR structure, e.g., a GPCR structure involved in binding one or more agonists. In some embodiments, an antagonist competes with one or more agonists of the GPCR for binding to the GPCR. In some embodiments, binding of an antagonist to a GPCR in the absence of an agonist binding to the GPCR does not affect an observed signaling activity of the GPCR.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biased agonist: As used herein, a “biased agonist” is a ligand that shows differential activity with respect to stimulation or activation of signaling by a GPCR that participates in a plurality of different signaling pathways. In some embodiments, a biased agonist interacts with all or part of a GPCR structure, e.g., a GPCR structure involved in binding an agonist. In some embodiments, a biased agonist competes with one or more agonists for binding to the GPCR. In some embodiments, binding by a biased agonist stabilizes a subset of possible GPCR conformations (e.g., conformations that promote and/or participate in signaling).

Inverse agonist: As used herein, an “inverse agonist” is a ligand that binds to a GPCR so that a decrease in signaling activity is observed relative to that of the GPCR in an unliganded (unbound state). In some embodiments, an inverse agonist interacts with all or part of a GPCR structure, e.g., a GPCR structure involved in binding one or more naturally-occurring (e.g., reference) ligands that regulate GPCR activity in vivo. In some embodiments, an inverse agonist competes with one or more naturally-occurring (e.g., reference) ligands of the GPCR for binding to the GPCR. In some embodiments, binding by an inverse agonist stabilizes one or more GPCR conformations (e.g., conformations that inhibit signaling).

Polypeptide: As used herein, a “polypeptide” is a polymer comprising at least two amino acid entities attached to one another by a peptide bond. In some embodiments, a polypeptide includes at least 3-5 amino acid entities, each of which is attached to others by way of at least one peptide bond. In some embodiments, a polypeptide is or comprises a prepropolypeptide, propolypeptide or prepolypeptide. In some embodiments, a polypeptide is or comprises a polypeptide chain having an amino acid sequence identical to that of a protein that is naturally produced by a cell. In some embodiments, a polypeptide is or comprises a polypeptide chain having an amino acid sequence that is not identical to that of any protein known to be produced by a cell as of the effective date of the present disclosure. In some embodiments, a polypeptide is produced by a cell. In some embodiments, a polypeptide is produced using chemical synthesis.

Receptorome: A “receptorome”, as used herein, is a collection of different GPCRs from an organism or from a region of an organism. In some embodiments, a receptorome is or includes all or substantially all known GPCRs from an organism or from a region of an organism. In some embodiments, a receptorome is or includes all or substantially all GPCRs from an organism or from a region of an organism known to bind to naturally-occurring ligands. In some embodiments, a receptorome is or includes at least about 70%, at least about 75%, at least about 80%, at least about 85%, at least about 90%, at least about 95%, or 100% of all known GPCRs from an organism or a region of an organism. In some embodiments, a receptorome is or includes at least about 200, at least about 225, at least about 250, at least about 275, at least about 300, at least about 325, at least about 350, at least about 375, at least about 400, at least about 425, at least about 450, at least about 475, at least about 500, at least about 525, at least about 550, at least about 575, at least 600, or more different GPCRs from an organism or a region of an organism. In some embodiments, a receptorome is or includes a mammalian, e.g., human or non-human primate, receptorome. In some embodiments, a receptorome is or includes all or substantially all known GPCRs from an organ or a tissue of an organism, e.g., a brain, heart, nose, etc.

Reference: A “reference” entity, system, amount, set of conditions, etc., is one against which a test entity, system, amount, set of conditions, etc. is compared as described herein. For example, in some embodiments, a “reference” individual is a control individual who is not suffering from or susceptible a disease, disorder or condition; in some embodiments, a “reference” individual is a control individual afflicted with the same form of disease, disorder or condition as an individual being treated, and optionally who is about the same age as the individual being treated (to ensure that the stages of the disease in the treated individual and the control individual(s) are comparable). In some embodiments, a “reference” ligand is a naturally-occurring ligand, control agonist, control antagonist, control inverse agonist, or control biased agonist that binds a GPCR. In some embodiments, binding of a test agent to GPCRs on a microarray is compared to that of a reference ligand.

Subject: As used herein, the term “subject”, “individual”, or “patient” refers to any organism upon which embodiments of the invention may be used or administered, e.g., for experimental, diagnostic, prophylactic, and/or therapeutic purposes. In some embodiments, a subject is a mammal, e.g., a human or non-human primate (e.g., an ape, monkey, orangutan, or chimpanzee), a dog, cat, guinea pig, rabbit, rat, mouse, horse, cattle, or cow.

Therapeutic agent: As used herein, the phrase “therapeutic agent” refers to any agent that, when administered to a subject, has a therapeutic effect and/or elicits a desired biological and/or pharmacological effect.

### DETAILED DESCRIPTION

In a human, there are about 400 known different G protein coupled receptors (GPCRs) for endogenous ligands that regulate virtually every physiological process by mediating effects of various hormones, neurotransmitters, peptides, metabolites, nucleotides and ions. Many currently used drugs act through GPCRs. A current paradigm in pharmacology is that such drugs produce therapeutic effects based on their interactions with specific receptors. The current disclosure encompasses a novel view of actions of drugs on GPCRs, i.e., that certain drugs may produce their therapeutic effects by acting directly or indirectly through multiple GPCRs. The disclosure encompasses novel microarrays and methods to determine interactions of ligands (e.g., therapeutic agents) with GPCRs. In particular, the present disclosure encompasses methods of screening a microarray constituting a receptorome (Roth et al., Proc. Natl. Acad. Sci. USA 99:11934-11939 (2002); Armbruster et al., J. Biol. Chem. 280:5129-5132 (2005)) and/or screening a microarray to identify and/or characterize GPCR targets for agents, e.g., therapeutic agents. Determining such interactions is useful, e.g., in selecting an appropriate therapeutic drug for a particular disease, disorder, or condition.

### G Protein Coupled Receptors

G protein-coupled receptors (GPCRs) are known in the art, and constitute a large and diverse superfamily of cell surface receptors. Approximately 800 distinct genes encoding functional GPCRs make up greater than 1% of the human genome (Lander et al., Nature 409:860-921 (2001); Venter et al., Science 291:1304-1351 (2001)). With alternative splicing, it is estimated that 1000 to 2000 discrete receptor proteins can be expressed. Olfactory-specific GPCRs include a family of GPCRs encoded by about 700 genes in humans to over about 1,200 genes in rodents (see, e.g., DeMaria et al., J. Cell Biol. 191:443-452 (2010)). Sequence similarities, hydropathy plots, and biochemical and mutagenic data support the conclusion that GPCRs share a common seven transmembrane domain architecture. Transmembrane domains share the highest degree of sequence conservation, while intracellular and extracellular domains exhibit extensive variability in size and complexity. Extracellular and transmembrane regions of GPCRs are involved in ligand binding, while intracellular domains are important for signal transduction and for feedback modulation of receptor function.

Classification systems have been devised that group GPCRs based upon their ligands or sequence similarities. Kolakowski's A through F classification system (Kolakowski, Recept. Channels 2:1-7 (1994)), for example, divides GPCRs into six families, of which three (Families A, B, and C) contain a majority of known human receptors. In this system, Family A is made up of rhodopsin-related receptors and is the largest group, containing receptors for biogenic amines and other small nonpeptide ligands, chemokines, opioids and other small peptides, protease-activated receptors, and receptors for glycoprotein hormones. Family B GPCRs, the second largest group, contains receptors that bind to higher-molecular-weight peptide hormones, such as glucagon, calcitonin and parathyroid hormone. Family C, the smallest group, contains metabotropic glutamate receptors, GABA_{B} receptor, and calcium-sensing receptor. Another classification system (GRAFS) groups GPCRs into five families: Glutamate, Rhodopsin, Adhesion, Frizzled/Taste2, and Secretin (Fredriksson, Mol. Pharmacol. 63:1256-1272 (2003)).

GPCRs function as ligand-activated guanine nucleotide exchange factors (GEFs) for heterotrimeric G proteins. The binding of a first messenger ligand to an extracellular or transmembrane domain of a GPCR triggers conformational changes that are transmitted through intracellular domains to promote coupling between a GPCR and its cognate G protein(s). A GPCR stimulates G protein activation by catalyzing exchange of GTP for GDP on the Gα subunit and dissociation of GTP-bound Gα subunit from Gβγ subunit heterodimer. Once dissociated, free Gα-GTP and Gβγ subunits regulate activity of enzymatic effectors, such as adenylate cyclases, phospholipase Cβ isoforms, and ion channels to generate small molecule second messengers. Second messengers, in turn, control activity of protein kinases that regulate enzymes involved in intermediary metabolism. Signaling continues until intrinsic GTPase activity of a Gα subunit returns a G protein to an inactive heterotrimeric state. In some instances, two broad signaling branches flow from an activated GPCR, including a β-arrestin branch and a G protein branch (see, e.g., Magalhaes et al., Br. J. Pharmacol. 165:1717-1736 (2012)).

### Microarrays

The present disclosure encompasses microarrays of GPCRs, and the use of such microarrays, e.g., to identify interactions of ligands with GPCRs. In some embodiments, microarrays of the present disclosure are used to analyze simultaneously effects of a test agent (e.g., a therapeutic drug) on all known human GPCRs for endogenous ligands (the “receptorome”) in a single assay. Receptorome screening is useful in identifying, characterizing, and/or predicting effects (e.g., biological activities, salutary effects, side effects, etc) (see, e.g., Setola, Drug News Perspect. 22:459-466 (2009)).

In some embodiments, microarrays of the present disclosure are used to measure (e.g., directly measure) activation states of GPCRs in response to a test agent. In some embodiments, microarrays of the present disclosure are used to identify a test agent as an agonist, an antagonist, a biased agonist, or an inverse agonist, of one or more GPCRs.

Microarrays of the present disclosure generally include a substrate having a surface to which GPCRs are attached, in spatially discrete locations. In some embodiments, such spatially discrete locations are arranged in a pattern on a substrate surface. In some embodiments, a spatially distinct location on a microarray is referred to herein as a “spot”. In general, spots can be any shape, such as circular, elliptoid, oval, annular, or another analogously curved shape.

In some embodiments, a microarray of the present disclosure includes at least about 100, at least about 200, at least about 300, at least about 400, at least about 500, at least about 600, at least about 700, at least about 800, at least about 900, at least about 1000, at least about 1250, at least about 1500, at least about 1750, at least about 2000, at least about 3000, at least about 4000, at least about 5000, at least about 6000, at least about 7000, at least about 8000, at least about 9000, at least about 10000, or more, spots.

In some embodiments, a microarray of the present disclosure includes at least about 1 spot/cm² of substrate, at least about 10 spots/cm² of substrate, at least about 20 spots/cm² of substrate, at least about 30 spots/cm² of substrate, at least about 40 spots/cm² of substrate, at least about 50 spots/cm² of substrate, at least about 60 spots/cm² of substrate, at least about 70 spots/cm² of substrate, at least about 80 spots/cm² of substrate, at least about 90 spots/cm² of substrate, at least about 100 spots/cm² of substrate, at least about 150 spots/cm² of substrate, at least about 200 spots/cm² of substrate, at least about 250 spots/cm² of substrate, at least about 300 spots/cm² of substrate, at least about 350 spots/cm² of substrate, at least about 400 spots/cm² of substrate, at least about 450 spots/cm² of substrate, at least about 500 spots/cm² of substrate, at least about 550 spots/cm² of substrate, at least about 600 spots/cm² of substrate, at least about 650 spots/cm² of substrate, at least about 700 spots/cm² of substrate, at least about 850 spots/cm² of substrate, at least about 900 spots/cm² of substrate, at least about 950 spots/cm² of substrate, at least about 1000 spots/cm² of substrate, or more.

In some embodiments, a microarray described herein includes spots arranged in rows and columns so as to form a grid, in a circular pattern, or any other convenient arrangement across the surface of the substrate.

In some embodiments, each spot of a microarray includes only one known type of GPCR. In some embodiments, each spot of a microarray includes more than one known type of GPCR. In some embodiments, a spot can include one or more effectors and/or adaptors of GPCRs described herein, e.g., G proteins, and/or β-arrestin polypeptides.

In some embodiments, one or more GPCRs is present in a spot together with or in context of a particle such as a lipid or polymer particle. In some embodiments, such a lipid or polymer particle is a synthetic or naturally occurring lipid or polymer particle. For example, such lipid or polymer particles include, but are not limited to, vesicles, liposomes, monolayer lipid membranes, bilayer-lipid membranes, membranes incorporated with receptors, whole or part of cell membranes, or liposomes containing re-folded proteins, or detergent micelles containing re-folded proteins, or the like. Membranes suitable for use with microarrays of the disclosure include, for example, but are not limited to, phospholipids, sphingomyelins, cholesterol or their derivatives.

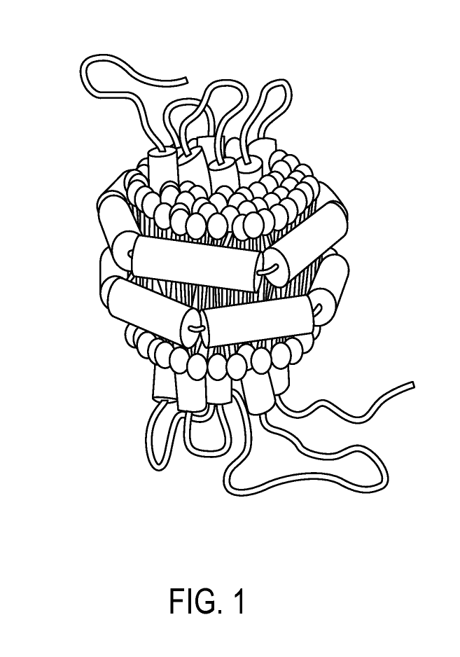

In some embodiments, a GPCR is included in a nanodisc or a nanolipid particle to provide a membrane-like environment. Nanodiscs or nanolipoprotein particles are discoidal phospholipid bilayers surrounded by apolipoproteins (see, e.g., Bayburt et al., Nano Letters 2:853-856 (2002)). FIG. 1 depicts an exemplary nanodisc useful in microarrays of the disclosure. As shown in FIG. 1, a nanodisc can include a lipid bilayer surrounded by two molecules of apolipoproteins.

In some embodiments, a GPCR present in a particular first spot differs from a GPCR present in a second spot of a microarray.

In some embodiments, a microarray includes at least about 50, at least about 100, at least about 150, at least about 200, at least about 250 at least about 300, at least about 350, at least about 400, at least about 450, at least about 500, at least about 550, at least about 600, at least about 650, at least about 700, at least about 750, or more, different GPCRs. In some embodiments, a microarray includes all or almost all known GPCRs, e.g., all or almost all known human GPCRs.

In some embodiments, a microarray includes at least about 50, at least about 100, at least about 150, at least about 200, at least about 250 at least about 300, at least about 350, at least about 400, at least about 450, at least about 500, at least about 550, at least about 600, at least about 650, at least about 700, at least about 750, or more, different olfactory GPCRs. In some embodiments, a microarray includes all or almost all known olfactory receptors of an organism.

In some embodiments, each spot of a microarray includes a different GPCR (e.g., each unique GPCR is included in only one spot of a microarray). In some embodiments, a microarray includes a unique GPCR in more than one spot. For example, a unique GPCR can be included in at least 2, 3, 4, 5, 6, 7, 8, 9, 10, or more, spots.

A variety of substrates useful in arrays and microarrays are known, and any substrate can be used in a microarray of the present disclosure. A suitable substrate can be formed from or comprise, e.g., without limitation, a ceramic substance, glass, metal, crystalline material, plastic, polymer or co-polymer, any combinations thereof, or a coating of one material on another. Such substrates include for example, but are not limited to, (semi) noble metals such as gold or silver; glass materials such as soda-lime glass, pyrex glass, vycor glass, quartz glass; metallic or non-metallic oxides; silicon, monoammonium phosphate, and other such crystalline materials; transition metals; plastics or polymers, including dendritic polymers, such as poly(vinyl chloride), poly(vinyl alcohol), poly(methyl methacrylate), poly(vinyl acetate-maleic anhydride), poly(dimethylsiloxane)monomethacrylate, polystyrenes, polypropylene, polyethyleneimine; copolymers such as poly(vinyl acetate-co-maleic anhydride), poly(styrene-co-maleic anhydride), poly(ethylene-co-acrylic acid) or derivatives of these or the like.

Substrates of a microarray described herein can have any of a variety of configurations, from simple to complex, e.g., an overall slide or plate configuration, such as a rectangular or disc configuration. In some embodiments, a microarray has or includes a standard microplate configuration. In some embodiments, a substrate has a rectangular cross-sectional shape, having a length of from about 10 mm to 200 mm, from about 40 to 150 mm, or from about 75 to 125 mm. In some embodiments, a substrate has a width of from about 10 mm to 200 mm, from about 20 mm to 120 mm, or from about 25 to 80 mm. In some embodiments, a substrate has a thickness of from about 0.01 mm to 5.0 mm, from about 0.1 mm to 2 mm, or from about 0.2 to 1 mm.

In some embodiments, spots are associated, e.g., stably associated, with a particular (e.g., a top) surface of a substrate. By “stably associated” is meant that spots maintain their position relative to a substrate under various conditions, for example during assay conditions described herein. Spots of GPCRs, e.g., spots of lipid membranes or lipid particles including GPCRs, can be non-covalently or covalently associated, e.g., stably associated, with a top surface of a substrate. Examples of non-covalent association include, without limitation, non-specific adsorption, binding based on electrostatic (e.g., ion, ion pair interactions), hydrophobic interactions, hydrogen bonding interactions, surface hydration force and the like, and specific binding based on specific interaction of an immobilized binding partner and a membrane bound protein. Specific types of binding-induced immobilization include, for example, antibody-antigen interaction, generic ligand-receptor binding, lectin-sugar moiety interaction, etc. Examples of covalent association include, but are not limited to, covalent bonds formed between a spot (e.g., a GPCR or a lipid membrane or lipid particle including a GPCR) and a functional group present on a top surface of a substrate. In some embodiments, a functional group is introduced onto a top surface of a substrate using a coating material. In some embodiments, a functional group present on a top surface of a substrate is NH₂. In some embodiments, GPCRs include histidine tags and a top surface of a substrate includes a Ni-presenting coating material.

In some embodiments, a substrate includes a coating material, e.g., on all or a part of a substrate, e.g., on a particular (e.g., top) surface of a substrate. In some embodiments, a coating material is or includes a silane, thiol, disulfide, or polymer. In some particular embodiments, a microarray includes a glass substrate and a silane coating material, e.g., a silane that presents terminal moieties including, for example, hydroxyl, carboxyl, phosphate, glycidoxy, sulfonate, isocyanato, thiol, or amino groups. In some embodiments, a coating material is or includes a silane, such as gamma-aminopropyl silane (GAPS) (see, e.g., Hong et al., J. Am. Chem. Soc. 127:15350-15351 (2005)). Alternative or additional coating materials are described, for example, in, US Patent Publ. No. 20100184626.

In some embodiments, a substrate is or includes a gold-coated surface and a coating material is or includes a thiol, e.g., a thiol comprising hydrophobic and hydrophilic moieties. In some embodiments, a coating material is a thioalkyl compound such as, but not limited to a thioalkyl acid (e.g., 16-mercaptohexadecanoic acid), thioalkyl alcohol, thioalkyl amine, or a halogen containing thioalkyl compound. Such compounds can be readily synthesized using known methods and/or can be obtained from commercial sources.

In some embodiments, a coating material is or includes a derivatized monolayer or multilayer having one or more covalently bonded linker moieties described herein. For example, a monolayer coating can include long chain hydrocarbon moieties having, but not limited to, thiol (e.g., thioalkyl), disulfide or silane groups (e.g., that produce a chemical or physicochemical bonding to a substrate). Additionally or alternatively, a monolayer can be attached to a substrate covalently or non-covalently.

In some embodiments, a monolayer coating material includes one or more reactive functional groups. Examples of reactive functional groups include, but are not limited to, carboxyl, isocyanate, halogen, amine and hydroxyl groups. In some embodiments, a monolayer coating material includes one or more activated reactive functional groups. Examples of activated reactive functional groups include, but are not limited to, anhydrides, N-hydroxysuccinimide esters, and additional known activated esters or acid halides (such as for covalent coupling to terminal amino groups of a linker moiety). In some embodiments, activated functional groups on a monolayer coating are or include anhydride derivatives (e.g., for coupling with a terminal hydroxyl group of a linker moiety); hydrazine derivatives (e.g., for coupling onto oxidized sugar residues of a linker moiety); or maleimide derivatives (e.g., for covalent attachment to thiol groups of a linker moiety). In some embodiments, a derivatized monolayer coating is produced by activating at least one terminal carboxyl group on a monolayer coating to an anhydride group and then reacting with a linker moiety.

In some embodiments, one or more reactive functional groups on a monolayer coating are reacted with a linker moiety having activated functional groups, for example, but not limited to, N-hydroxysuccinimide esters, acid halides, anhydrides, and isocyonates for covalent coupling to reactive amino groups on a monolayer coating.

In some embodiments, a linker moiety includes a terminal functional group, a spacer region, and a GPCR adhering region (e.g., a region that adheres to a lipid membrane or a lipid particle including a GPCR). In some embodiments, a terminal functional group is a carboxylic acid, halogen, amine, thiol, alkene, acrylate, anhydride, ester, acid halide, isocyanate, hydrazine, maleimide or hydroxyl group. In some embodiments, a terminal functional group of a linker moiety is reacted with an activated functional group on a monolayer coating.

In some embodiments, a spacer region is or includes an oligo/polyether, oligo/polypeptide, oligo/polyamide, oligo/polyamine, oligo/polyester, oligo/polysaccharide, polyols, multiple charged species, or a combination thereof. Examples include, but are not limited to, oligomers of ethylene glycols, peptides, glycerol, ethanolamine, serine, inositol, etc. In some embodiments, a spacer region is or includes a hydrophilic spacer region. In one embodiment, a spacer region has or includes between about 2 and about 25 oxyethylene groups.

In some embodiments, a GPCR adhering region is or includes a hydrophobic or amphiphilic tail of a linker moiety. In some embodiments, a hydrophobic or amphiphilic tail is or includes a straight or branched chain alkyl, alkynyl, alkenyl, aryl, araalkyl, heteroalkyl, heteroalkynyl, heteroalkenyl, heteroaryl, or heteroaraalkyl. In one embodiment, a GPCR adhering region is or includes a C₁₀ to C₂₅ straight or branched chain alkyl or heteroalkyl hydrophobic tail. In some embodiments, a linker moiety includes a terminal functional group at a first end, a spacer, a linker/GPCR adhering region, and a hydrophilic group at a second end.

In some embodiments, a derivatized monolayer is or includes an alkanethiol modified with a silane (e.g., a self-assembled monolayer (SAM) of an alkanethiol modified with a silane). Exemplary alkanethiols include, without limitation, 11-mercaptoundecanol (MUD), 11-mercaptoundecanoic acid (MUA), 11-mercaptoundecylamine (MUAM), 16-mercaptohexadecanol, and 16-mercaptohexadecanoic acid. Silanes include, e.g., 3-aminopropyltrimethoxysilane (APTES), 3-mercaptopropyltrimethoxysilane, and 3-isocyanatopropyltriethoxysilane.

### Production of GPCRs

Microarrays of the present disclosure include GPCRs attached to substrates as described herein. In some embodiments, microarrays include spots of purified GPCRs. In some embodiments, GPCRs are incorporated into lipid membranes and/or lipid particles.

In some embodiments, a GPCR includes one or more markers, e.g., one or more detectable markers. In some embodiments, such one or more markers produce a measurable and/or detectable signal, e.g., a detectable signal correlated to a conformational state of a GPCR. For example, a GPCR can include a first and a second marker that, in combination, produce a measurable and/or detectable difference in an overall signal (“marker signal”) of such first and second markers between a first and a second conformation state of a GPCR. In some embodiments, a first and second marker produce a first marker signal in a first GPCR conformation state that is a different (e.g., a detectably different) signal than a second marker signal produced in a second conformation state. In some embodiments, a first and a second marker produce a weak or no marker signal (i.e., an undetectable signal) in a first GPCR conformation state. In some embodiments, a first and a second marker produce a strong marker signal in a second GPCR conformation state. In some embodiments, such a difference in marker signals is higher than a control signal (e.g., a background signal). In some embodiments, a difference in marker signals between a first and a second GPCR conformation state is at least about 2, 3, 4, 5, 6, 7, 8, 9, 10, or more times higher than a control signal (e.g., a background signal).

In some embodiments, markers are or include fluorescent markers, e.g., small or medium sized fluorescence molecules, fluorescent proteins, or fluorescent peptides. In some embodiments, markers are spatially sensitive markers, e.g., fluorescence resonance energy transfer (FRET) markers, bioluminescence resonance energy transfer (BRET) markers, or luminescence resonance energy transfer (LRET) markers. For example, a spatially sensitive marker can utilize a spatial proximity to one or more additional spatially sensitive markers to produce a marker signal. In some embodiments, a first and a second spatially sensitive marker produce a marker signal when located in a spatial proximity of less than about 10 nm, less than about 9 nm, less than about 8 nm, less than about 7 nm, less than about 6 nm, less than about 5 nm, less than about 4 nm, less than about 3 nm, less than about 2 nm, or less than about 1 nm, of each other. Spatially sensitive markers are known in the art and include, without limitation, variants of green fluorescent protein (GFP), chemical fluorophores or chromophores such as tetramethylrhodamine, fluoresceine, 5,5′-dithiobis-(2-nitro)-benzoic acid, or thiol-reactive dyes or derivatives thereof. Additional or alternative spatially sensitive markers include, e.g., cyan fluorescent protein (CFP) and fluorescein arsenical hairpin binder (FlAsH label) (see, e.g., Vilardaga, Methods Mol. Biol. 756:133-148 (2011); Zurn et al., Mol. Pharmacol. 75:534-541 (2009)).

In some embodiments, a marker is a naturally expressed marker. In some embodiments, a marker is a synthetic marker. In some embodiments, a marker is associated with a GPCRs by a covalent bond. In some embodiments, a marker is associated with a GPCR by an affinity, ionic, van der Waals, or hydrophobic association. In some embodiments, a microarray described herein includes fusion proteins that encompass GPCRs and one or more markers, e.g., one or more spatially sensitive markers.

In some embodiments, GPCRs include markers at one or more locations within a GPCR, e.g., one or more locations that undergo conformation changes based on activation state of a GPCR. Crystal structures of active and inactive functional states of GPCRs are known (Rasmussen et al., Nature 477:549-555 (2011); Katritch et al., Trends Pharmacol. Sci. 33:17-27 (2012)). Analysis of these structures indicates that receptor activation can be associated with changes in positioning of transmembrane (TM) helices, and that binding of an agonist ligand can lead to rotation of TM6. An intracellular segment of TM6 tilts outward, away from the receptor center, thereby opening the receptor for binding of an active form of a G protein. The displacement of TM6 is approximately 14 Å relative to other TM helices (Rasmussen et al., Nature 477:549-555 (2011)). In some embodiments, a GPCR includes a marker within or near TM6.

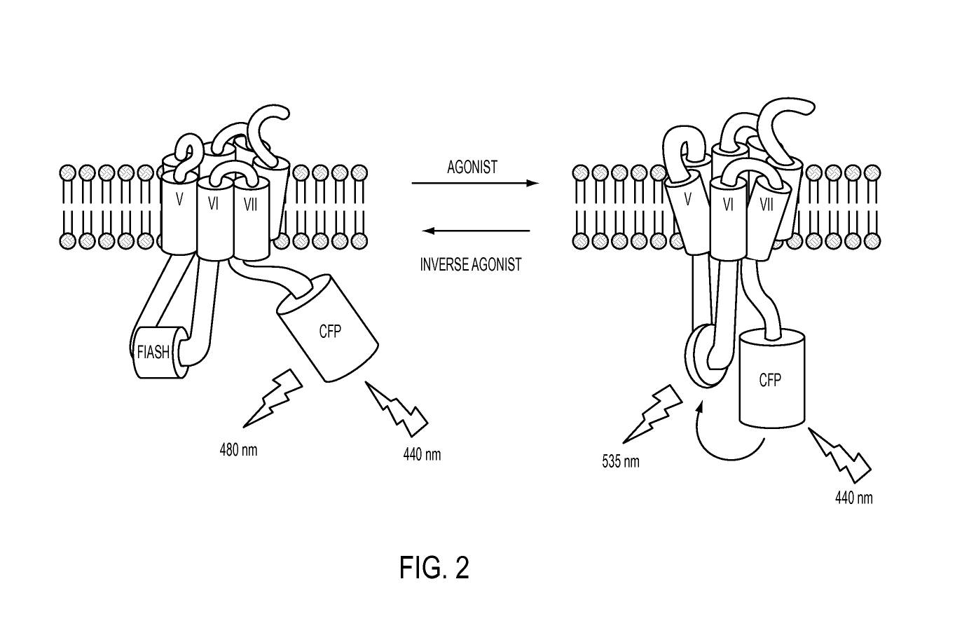

FIG. 2 depicts an exemplary GPCR having markers at two locations with the GPCR. As shown in FIG. 2, a first marker (CFP) is fused to a C-terminus of a GPCR, and a second marker (FlAsH label) placed within intracellular loop 3 (ICL3) of a GPCR, which is N-terminal to transmembrane helix 6 (TM6). FlAsH label can be added to ICL3 by insertion of a tetracysteine motif (CCXXCC, such as CCPGCC) into an amino acid sequence of a GPCR within ICL3 (see, e.g., Vilardaga, Methods Mol. Biol. 756:133-148 (2011); Adams et al., J. Am. Chem. Soc. 124:6063-6076 (2002)). In the exemplary method of FIG. 2, activation of a GPCR can be visualized by the ratio of 480 nm (donor fluorescence of CFP) and 535 nm (FRET emission of FlAsH) emission upon excitation of CFP at 440 nm.

Certain models of GPCRs suggest that GPCRs exist in multiple states, where agonists induce distinct active conformations of GPCRs by exposing different regions of intracellular domains of GPCRs involved in coupling to different G protein populations (see, e.g., Seifert et al., Mol. Pharmacol. 56:348-358 (1999); Maudsley et al., J. Pharm. Exp. Therapeutics 314: 485-494 (2005)). Different areas of intracellular loops on GPCRs are known to activate different G proteins (see, e.g., Wade et al., Mol. Pharmacol. 56:1005-1013 (1999)). Biased agonists can produce distinct tertiary conformations of a GPCR, exposing particular intracellular regions to allow biased activation of certain G proteins. In some embodiments, a microarray includes GPCRs including a marker described herein within different intracellular loops of GPCRs. For example, a microarray can include multiple spots of a particular GPCR, each particular GPCR including a marker located at different intracellular loops of the GPCR. In some embodiments, a GPCR includes a second marker at its C-terminus. Such microarrays are useful in identifying biased agonists of GPCRs.

GPCRs can be obtained or produced by any of a variety of means known to those of ordinary skill in the art. For examples, GPCRs can be obtained from natural sources, can be produced in cells using recombinant DNA methods, or can be produced synthetically, e.g., using cell-free methods.

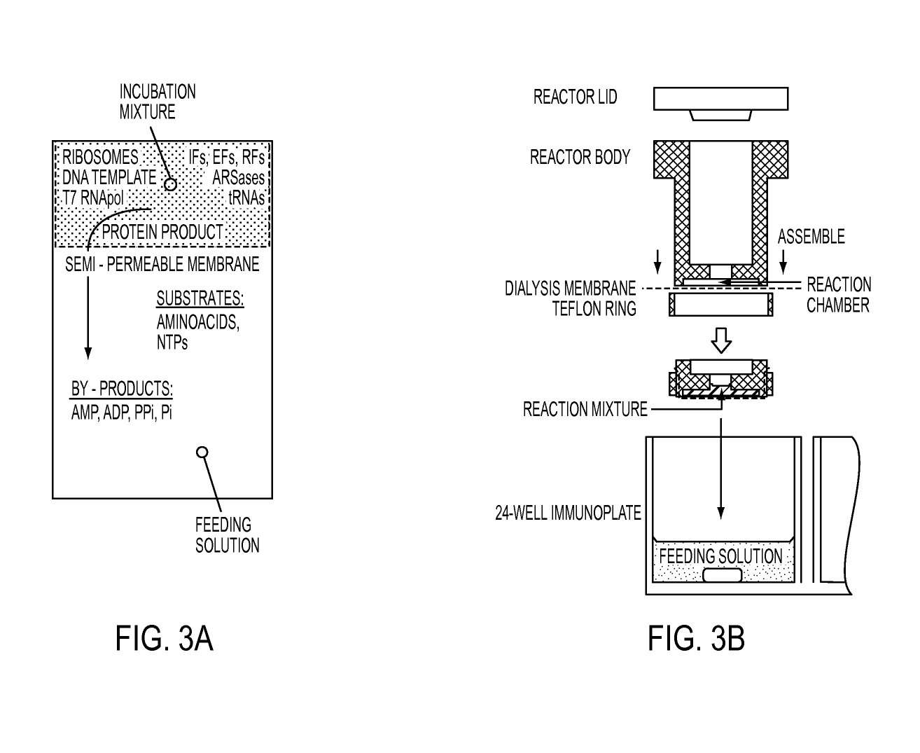

In some embodiments, GPCRs are produced using a cell-free method. In some embodiments, a coupled transcription/translation system is used, such as a system described by, e.g., Spirin et al., Science 242:1162-1164 (1988); Shirokov et al., Methods Mol. Biol. 375:19-55 (2007); Corin et al., PLoS One 6:e23036 (2011); or Klammt et al., Protein Sci. 20:1030-1041 (2011). In one embodiment, a continuous-exchange cell-free (CECF) protein synthesizing system is used. FIG. 3 depicts an exemplary system, which includes two compartments separated by a semi-permeable membrane. A larger compartment contains a feeding mixture, which supplies low molecular weight components, such as nucleotides and amino acids, for a reaction mixture. Lower molecular weight products produced by a reaction are dialysed into an external solution, while reaction components are continuously replenished in a reaction mixture.

In some embodiments, initial optimization of expression conditions are performed in small reactors, e.g., of a size suitable for analytical or micro-scale protein production (e.g., in about 50 μL to about 100 μL reaction mixtures, e.g., yielding about 50 μg to about 200 μg of protein product). In some embodiments, GPCRs are produced using dialysis cassettes (e.g., Slide-A-Lyzer Cassettes, Thermo Fisher Scientific, Rockford, Ill.).

In some embodiments, mutants or modifications of such proteins can be included in a microarray described herein. For example, some GPCRs possessing single or multiple point mutations retain biological functionality and may be involved in disease (see, e.g., Stadel et al., Trends Pharmocol. Rev. 18:430-437 (1997)).

In some embodiments, one or more GPCRs is present on a microarray together with or in context of a particle such as a lipid or polymer particle. In some embodiments, such lipid or polymer particles are produced using methods known in the art. For example, a GPCR can be inserted into a nanodisc cotranslationally or transferred from a detergent micelle, such as described by Yang et al., BMC Biotechnol. 11:57 (2011).

In some embodiments, GPCRs are produced by in vitro translation, for example in the presence of a mild detergent (e.g., Brij-35, Thermo Fisher Scientific, Rockford, Ill.) and/or a surfactant (e.g., NVoy, Expedeon, San Diego, Calif.) (see, e.g., Corin et al., PLoS One 6:e23036 (2011); Klammt et al., Protein Sci. 20:1030-1041 (2011)).

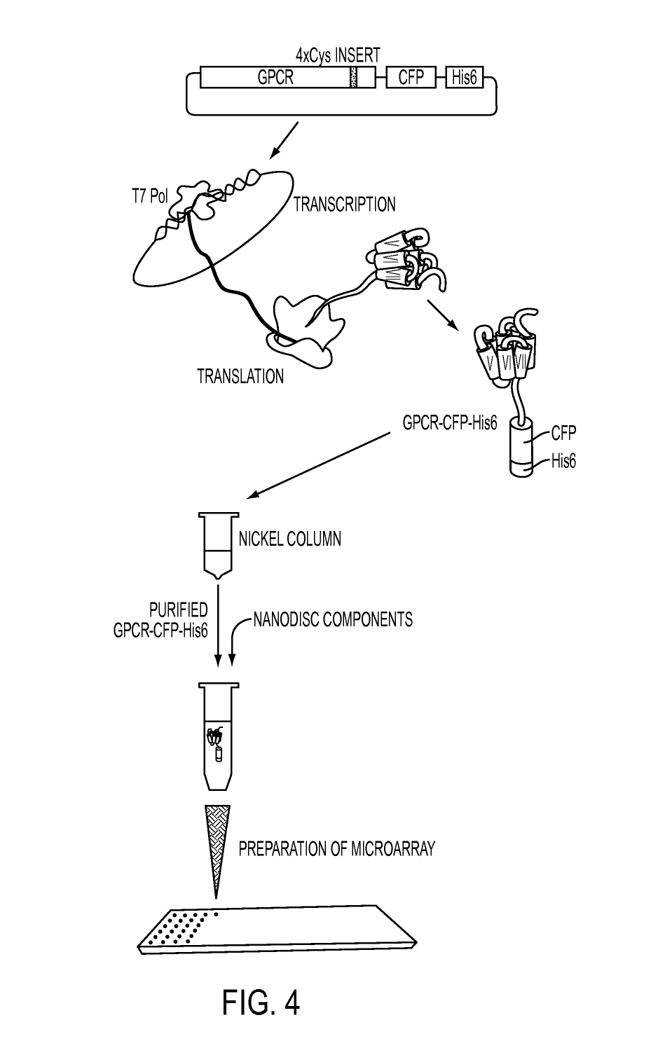

FIG. 4 depicts a particular exemplary method of GPCR expression in a cell-free system, and microarray preparation. As shown in FIG. 4, a gene construct is prepared that includes a gene encoding a GPCR containing a tetracysteine motif in ICL3, a sequence encoding a CFP protein, and a sequence encoding a His-tag at the C-terminus Expression of the gene construct produces a GPCR fusion protein including CFP and a His tag at a C-terminus. As depicted in FIG. 4, the gene construct is expressed in a constant exchange cell-free system including detergents, and GPCRs are purified on a immobilized nickel column. Purified GPCRs are then mixed with nanodisc components (lipids and apolipoproteins) to form nanodiscs containing GPCRs. Afterwards, GPCRs are applied to a substrate to produce a microarray.

In some embodiments, aspects of the invention comprise and/or utilize GPCRs together with one or more effectors, such as G proteins, G protein coupled receptor kinases (GRKs), or β arrestin.

In some embodiments, aspects of the invention comprise and/or utilize GPCRs, optionally together with one or more effectors, together with and/or in the context of a particle such as a lipid or polymer particle. In some embodiments, such particles comprise and/or are prepared from cell membranes from a cell line expressing a desired GPCR and its effectors. In some embodiments, a reconstituted GPCR in a liposome or micelle is used, for example in which a GPCR is associated with one or more effectors in a preferable ratio. A GPCR can be coupled with one or more effectors before or after being disposed on a microarray. Effectors can be, e.g., purified natural proteins or recombinant proteins.

In some embodiments, a GPCR includes a first marker, such as within a location of a GPCR that undergoes a conformation change based on activation state of a GPCR, and another protein, e.g., all or a portion of an effector protein, includes a second marker. In some embodiments, an effector protein is a G protein, e.g., an alpha subunit of a G protein, and such effector protein includes a second marker. In some embodiments, a portion of a G protein, e.g., all or a part of a carboxyl terminal alpha helix of a G alpha subunit, includes a second marker. Portions of G alpha subunits useful in methods described herein include, e.g., portions that bind to active conformations of GPCRs, e.g., those described by Rasmussen et al., Nature 477:549-555 (2011) and Choe et al., Nature 471:651-655 (2011).

### Microarray Preparation

Microarrays of the present disclosure can be prepared using any of a variety of known methods. In some embodiments, microarrays are prepared using micro-patterning techniques. For example, a tip of an applicator can be immersed into a solution containing GPCRs (e.g., lipid membranes or lipid nanoparticles containing GPCRs) and contacted with a top surface of a substrate to transfer the solution to the surface.

An applicator can be of any shape, size, or dimension. For example, a micro-patterning process may involve ring shaped applicators, square applicators, or point applicators, etc. In some embodiments, a micro-patterning technique utilizes a single applicator producing one or more spots containing the same type of GPCR. In some embodiments, a micro-patterning technique utilizes multiple applicators, e.g., configured in a pattern, and each applicator disposes a spot onto a substrate producing a microarray having or including spots configured in a similar pattern as the applicators.

A micro-patterning apparatus can include a print head, plate, substrate handling unit, XY or XYZ positioning stage, environmental control, instrument control software, sample tracking software, etc. A variety of other techniques can be used to produce a microarray of the disclosure including, for example, microstamping (see, e.g., U.S. Pat. No. 5,731,152), microcontact printing using PDMS stamps (see, e.g., Hovis et al., Langmuir 17:3400-3405 (2001)), capillary dispensing devices (see, e.g., U.S. Pat. No. 5,807,522), micropipetting devices (see, e.g., U.S. Pat. No. 5,601,980), and additional methods described in U.S. Patent Publ. 20100184626 and U.S. Patent Publ. 20090131263.

### Uses of Microarrays

Microarrays of the present disclosure are useful, for example, in drug development, medical diagnostics and proteomics. In some embodiments, one or more test agents is or are contacted with a microarray of the disclosure, and activation states of one or more GPCRs on the microarray are determined. In some embodiments, a single concentration of test agent is applied to a microarray; in some embodiments a plurality of concentrations of a test agent are applied. In some embodiments, a dose-response curve is generated or determined, for example by adding a test agent in more than one concentration and correlating response in GPCR activation state to test agent concentration. Furthermore, EC₅₀ or IC₅₀ of a test agent can be determined by identifying a concentration of a test agent that results in approximately 50% of maximum responses in GPCR activation state.

A test agent can be any sample of interest, including, e.g., a peptide, polypeptide, small molecule (organic or inorganic), or mixtures thereof. In some embodiments, a test agent is a pure preparation of a single particular entity such as a peptide, polypeptide, or small molecule. In some embodiments, a test agent is or comprises an impure preparation (i.e., a mixture of two or more entities such as peptides, polypeptides, and/or small molecules). For example, a test agent can be or comprise, or can be derived from, a biological sample from a subject, e.g., blood (including whole blood, plasma and serum), urine, cerebral spinal fluid, cells, or tissue.

In some embodiments, a microarray of the present disclosure is contacted with a therapeutic agent, and activation states of one or more GPCRs of the microarray are determined. In one embodiment, a biological sample is obtained from a subject who has been administered a therapeutic agent, a microarray of the present disclosure is contacted with the biological sample, and activation states of one or more GPCRs of the microarray are determined. In some embodiments, any therapeutic drug, whether or not known or suspected of interacting with a GPCR, can be contacted with a microarray of the present disclosure.

In some embodiments, activation states of a plurality of GPCRs (e.g., substantially all GPCRs) of a microarray of the present disclosure can be simultaneously determined. For example, a baseline measurement of GPCR activation states can be determined, e.g., by measuring marker signals from GPCRs of a microarray. Following baseline measurement, a test agent can be contacted with a microarray, and activation states of GPCRs of a microarray can be determined, e.g., by measuring marker signals from GPCRs of a microarray. By comparing activation states of GPCRs of a microarray before and after contact with a test agent, for example, effects of a test agent on activation states of GPCRs can be determined and/or characterized. In some embodiments, marker signals are based on FRET, as described herein.

In some embodiments, a microarray of the present disclosure is used to identify an agonist of one or more GPCRs. For example, a marker signal produced at a spot of a microarray following contact of a microarray with a test agent indicates the test agent is an agonist of a GPCR located at such spot of the microarray.

In some embodiments, a microarray of the present disclosure is used to identify an antagonist of one or more GPCRs. For example, a baseline measurement of activation states of GPCRs on a microarray can be determined A first agent, e.g., a first agent known to be an agonist of at least one GPCR on a microarray, is contacted with a microarray. Marker signals (e.g., FRET-based marker signals) from one or more spots are detected in the presence of the first agent. A test agent is then contacted with the microarray, e.g., in the presence of the first agent, and marker signals in the presence of the test agent are measured. A test agent that results in a diminished or absence of a marker signal from one or more spots from which a marker signal was detected in presence of the first agent, is identified as an antagonist of the one or more GPCRs located as such spots of the microarray.

In some embodiments, a microarray of the present disclosure is used to identify an inverse agonist of one or more GPCRs. For example, baseline (e.g., in the absence of a test agent) marker signals from spots on a microarray are measured. Following baseline measurement, a test agent is contacted with a microarray, and marker signals in presence of the test agent are measured. A test agent that results in a diminished or absence of a marker signal from one or more spots from which a marker signal was detected in the baseline measurement, is identified as an inverse agonist of the one or more GPCRs located as such spots of the microarray.

In some embodiments, a microarray of the present disclosure is used to diagnose a disease, condition, or disorder, in a subject. For example, a microarray described herein is contacted with a biological sample obtained from a test subject, and a test activation profile of GPCRs on the microarray in presence of the biological sample is determined A test activation profile is compared to a reference (e.g., a control activation profile, e.g., an activation profile from a control subject having a disease, condition, or disorder). A test activation profile that is similar to the control activation profile indicates the test subject has or is susceptible of developing such disease, condition, or disorder. For example, a test activation profile having at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more, of the same activation states of GPCRs of the control activation profile indicates the test subject has or is susceptible of developing such disease, condition, or disorder.

In some embodiments, a microarray of the present disclosure is contacted with a test agent, e.g., a drug, e.g., a therapeutic drug, and a first activation profile of GPCRs on the microarray is determined. In some embodiments, a microarray of the present disclosure is contacted with a biological sample from a subject having a disease, condition, or disorder, and a second activation profile of GPCRs on the microarray is determined A first activation profile that is similar to the second activation profile indicates the test agent (e.g., drug, e.g., therapeutic drug) causes or can increase incidence of the disease, condition, or disorder. For example, a first activation profile having at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more, of the same activation states of GPCRs of the second activation profile indicates the test agent (e.g., drug, e.g., therapeutic drug) causes or can increase incidence of the disease, condition, or disorder.

In some embodiments, a microarray of the present disclosure is contacted with a test agent, e.g., a drug, e.g., a therapeutic drug, and a first activation profile of GPCRs on the microarray is determined. In some embodiments, a microarray of the present disclosure is contacted with a biological sample from a subject having a disease, condition, or disorder, and a second activation profile of GPCRs on the microarray is determined A first activation profile that is similar to an inverse pattern of the second activation profile indicates the test agent (e.g., drug, e.g., therapeutic drug) is useful for treating or preventing the disease, condition, or disorder. For example, a first activation profile having at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more, of the same activation states of GPCRs of an inverse pattern of the second activation profile indicates the test agent (e.g., drug, e.g., therapeutic drug) is useful for treating or preventing the disease, condition, or disorder. In some embodiments, an inverse pattern of an activation profile is different from such activation pattern at one or more locations (e.g., an activation profile includes a GPCR in an activated state at one or more locations, and an inverse pattern includes a GPCR in a nonactivated state at such one or more locations).

In some embodiments, a microarray of the present disclosure includes olfactory GPCRs, and activation states of a plurality of olfactory GPCRs (e.g., substantially all olfactory GPCRs) of a microarray of the present disclosure can be simultaneously determined, e.g., to generate map of olfactory GPCR response to a test agent. For example, a baseline measurement of olfactory GPCR activation states can be determined, e.g., by measuring marker signals from olfactory GPCRs of a microarray. Following baseline measurement, a test agent can be contacted with a microarray, and activation states of olfactory GPCRs of a microarray can be determined, e.g., by measuring marker signals from olfactory GPCRs of a microarray. By comparing activation states of olfactory GPCRs of a microarray before and after contact with a test agent, for example, effects of a test agent on activation states of olfactory GPCRs can be determined and/or characterized. In some embodiments, marker signals are based on FRET, as described herein.

Marker signals can be measured using an analyzer, e.g., a scanner or reader, capable of detecting marker signals. For example, a scanner or reader capable of analyzing FRET-based microarrays can be used. In some embodiments, “staring” scanners can be used to detect presence or absence of marker signals from all, or substantially all, spots of a microarray simultaneously.

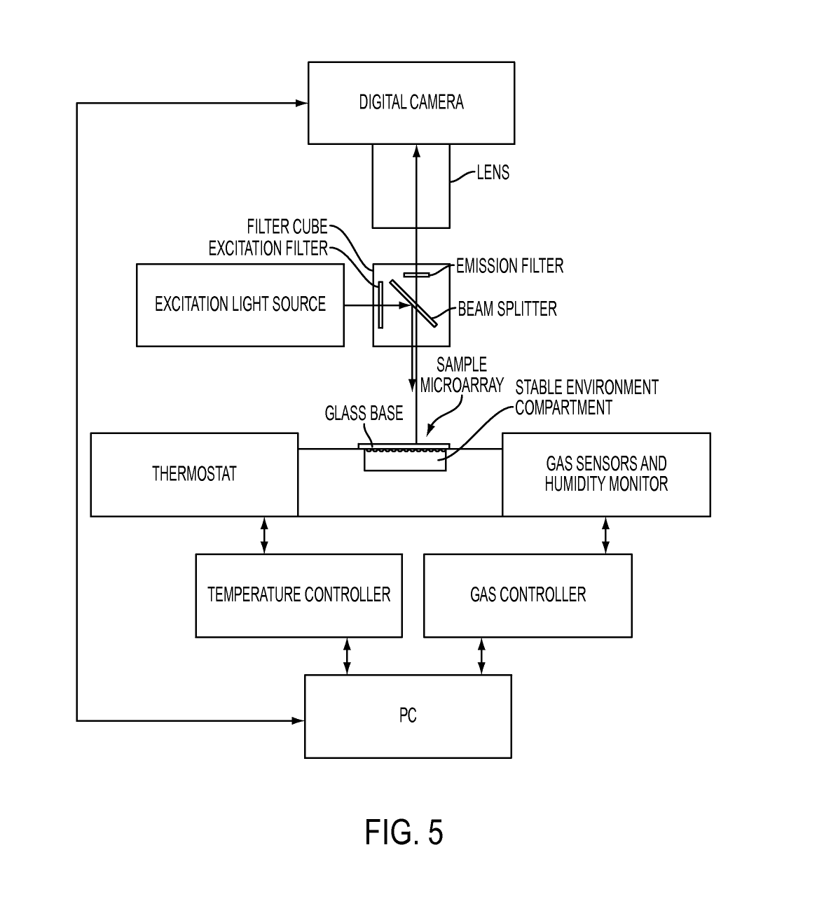

In some embodiments, an analyzer is based on a fluorescence microscope. An exemplary analyzer is schematically depicted in FIG. 5, and includes a light source, filter cube, lens and a camera. In some embodiments, an analyzer images an entire microarray, e.g., a microarray having dimensions up to about 10 mm×10 mm. In some embodiments, an analyzer images an entire microarray with a resolution of up to about 6.5 μm. A set of filters housed in a filter cube can be selected according to known optical properties of markers used in GPCRs of a microarray. As shown in FIG. 5, light from a blue solid state laser with uniform matrix beam splitter can pass through a clean-up filter and be reflected toward a microarray by a dichroic beam splitter. A marker signal (e.g., FRET-based light emitted from a GPCR in a microarray) can be transmitted through a dichroic beam splitter. An image of a microarray can be projected onto a CMOS sensor of a camera through an emitter filter. To facilitate acquisition of large numbers of snapshots for statistical analyses, an analyzer system can be automated and microarray ambient conditions (e.g., temperature, humidity and gas content) can be controlled.

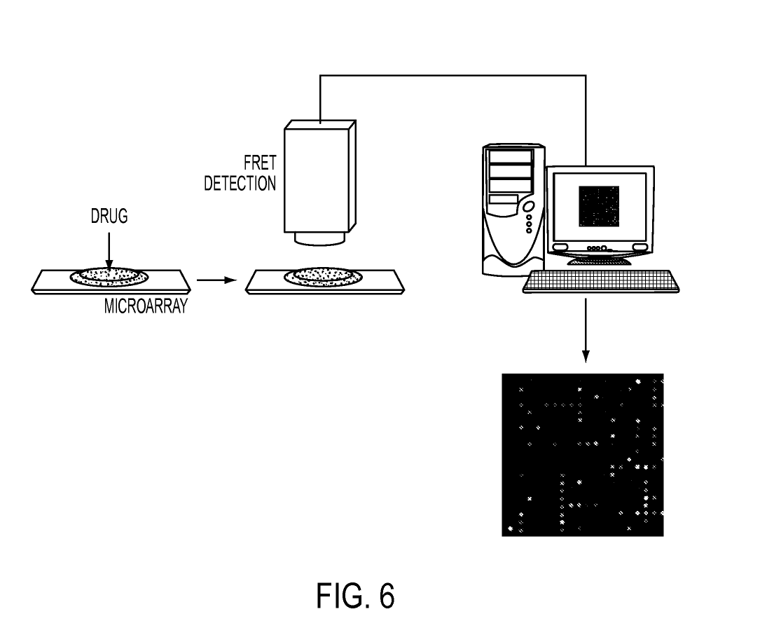

An exemplary method of analyzing effects of a drug on GPCR activation state is depicted schematically in FIG. 6. As shown in FIG. 6, a drug of interest is applied to a microarray containing GPCRs. FRET-based signals are simultaneously detected from GPCRs of the microarray, and signals are imaged to provide a pattern of GPCR activation states (e.g., activation profile) for a given drug.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A microarray comprising a substrate and at least about 400 unique G protein coupled receptors (GPCR) disposed on the substrate, wherein each GPCR is disposed at a predetermined location of the substrate, wherein each GPCR is linked to a first marker and a second marker, and wherein the first marker and the second marker produce a GPCR conformation dependent detectable signal.

2. The microarray of claim 1, wherein an inactive GPCR comprises an inactive conformation.

3. The microarray of any one of the preceding claims, wherein an activated GPCR comprises an active conformation.

4. The microarray of any one of the preceding claims, wherein binding of an activating ligand to a GPCR induces a conformational change of the GPCR from an inactive conformation to an active conformation.

5. The microarray of any one of the preceding claims, wherein the first marker and the second marker produce a detectable signal when the GPCR is in the active conformation.

6. The microarray of any one of the preceding claims, wherein the detectable signal is a fluorescence resonance energy transfer (FRET) signal, a bioluminescence resonance energy transfer (BRET) signal, or a luminescence resonance energy transfer (LRET) signal.

7. A method of producing a microarray, comprising:
providing at least about 400 different GPCRs, wherein each GPCR is linked to a first marker and a second marker, and wherein the first marker and the second marker produce a GPCR conformation dependent detectable signal; and
disposing each GPCR at a predetermined location of the substrate.

8. The method of claim 7, wherein each GPCR is provided as a lipid particle comprising the GPCR.

9. The method of claim 7 or 8, further comprising producing the GPCRs.

10. The method of any one of claims 7-9, further comprising producing the GPCRs using a cell-free method.

11. A method of identifying one or more GPCRs activated by a ligand, comprising:
providing a microarray comprising a substrate and at least about 400 different GPCRs disposed on the substrate, wherein each GPCR is disposed at a predetermined location of the substrate, wherein each GPCR is linked to a first marker and a second marker, and wherein the first marker and the second marker produce a GPCR conformation dependent detectable signal;
contacting the GPCRs with the ligand; and
measuring a presence or absence of a GPCR conformation dependent detectable signal at a predetermined location of the substrate, wherein a detectable signal measured at a predetermined location of the substrate identifies the GPCR at the predetermined location as a GPCR activated by the ligand.

12. The method of claim 11, wherein the presence or absence of the detectable signals at the predetermined locations are measured directly.

13. The method of claim 11 or 12, wherein the presence or absence of the detectable signals at the predetermined locations are measured simultaneously.

14. A method of identifying an agonist of one or more GPCRs, comprising:
providing a microarray comprising a substrate and at least about 400 different GPCRs disposed on the substrate, wherein each GPCR is disposed at a predetermined location of the substrate, wherein each GPCR is linked to a first marker and a second marker, and wherein the first marker and the second marker produce a GPCR conformation dependent detectable signal;
measuring an initial absence of a GPCR conformation dependent detectable signal at one or more predetermined locations of the substrate;
contacting the GPCRs with a test agent; and
measuring one or more GPCR conformation dependent detectable signals from the microarray in the presence of the test agent, wherein measurement of one or more GPCR conformation dependent detectable signals from the one or more predetermined locations of the substrate identifies the test agent as an agonist of a GPCR at the one or more predetermined locations.

15. A method of identifying an antagonist of one or more GPCRs, comprising:
providing a microarray comprising a substrate and at least about 400 different GPCRs disposed on the substrate, wherein each GPCR is disposed at a predetermined location of the substrate, wherein each GPCR is linked to a first marker and a second marker, and wherein the first marker and the second marker produce a GPCR conformation dependent detectable signal;
contacting the GPCRs with an agonist;
measuring an initial GPCR conformation dependent detectable signal at one or more predetermined locations of the substrate in the presence of the agonist;
contacting the GPCRs with a test agent; and
measuring an absence of one or more GPCR conformation dependent detectable signals from the microarray in the presence of the test agent, wherein measurement of an absence of one or more GPCR conformation dependent detectable signals from the one or more predetermined locations of the substrate identifies the test agent as an antagonist of a GPCR at the one or more predetermined locations.

16. A method of identifying an inverse agonist of one or more GPCRs, comprising:
providing a microarray comprising a substrate and at least about 400 different GPCRs disposed on the substrate, wherein each GPCR is disposed at a predetermined location of the substrate, wherein each GPCR is linked to a first marker and a second marker, and wherein the first marker and the second marker produce a GPCR conformation dependent detectable signal;
measuring an initial GPCR conformation dependent detectable signal at one or more predetermined locations of the substrate;
contacting the GPCRs with a test agent; and
measuring an absence of one or more GPCR conformation dependent detectable signals from the microarray in the presence of the test agent, wherein measurement of an absence of one or more GPCR conformation dependent detectable signals from the one or more predetermined locations of the substrate identifies the test agent as an inverse agonist of a GPCR at the one or more predetermined locations.

17. The method of any one of claims 7-16, wherein the detectable signal is a fluorescence resonance energy transfer (FRET) signal, a bioluminescence resonance energy transfer (BRET) signal, or a luminescence resonance energy transfer (LRET) signal.
